(19) United States
(12) Patent Application Publication
(10) Pub. No.: US 2024/0191174 A1
(43) Pub. Date: Jun. 13, 2024

(54) CELL CULTURE APPARATUS AND CELL CULTURE METHOD

(71) Applicants: CANON MEDICAL SYSTEMS CORPORATION, Otawara-shi (JP); Canon Kabushiki Kaisha, Tokyo (JP)

(72) Inventors: Mayumi SAKAGAMI, Ibaraki (JP); Koji HIRATA, Kasukabe (JP); Kanako AIZAWA, Yaita (JP)

(73) Assignees: CANON MEDICAL SYSTEMS CORPORATION, Otawara-shi (JP); Canon Kabushiki Kaisha, Tokyo (JP)

(21) Appl. No.: 18/533,617

(22) Filed: Dec. 8, 2023

(30) Foreign Application Priority Data
Dec. 9, 2022 (JP) 2022-197418

(51) Int. Cl. C12M 1/34

(52) U.S. Cl. CPC C12M 41/34; C12M 41/12

(57) ABSTRACT

A cell culture apparatus according to an embodiment includes a culture chamber, a holding unit, and processing circuitry. In the culture chamber, a biological specimen is cultured. The holding unit is provided in the culture chamber and configured to hold the biological specimen in such a manner that the position thereof in the culture chamber is movable. The processing circuitry is configured to control the holding unit so as to move the position of the biological specimen within the culture chamber, in a culture period for culturing the biological specimen.

### CROSS-REFERENCE TO RELATED APPLICATIONS

This application is based upon and claims the benefit of priority from Japanese Patent Application No. 2022-197418, filed on Dec. 9, 2022, the entire contents of which are incorporated herein by reference.

### FIELD

Embodiments described herein relate generally to a cell culture apparatus and a cell culture method.

### BACKGROUND

Stem cells such as induced Pluripotent Stem (iPS) cells and Embryonic Stem (ES) cells have capacity to differentiate into cells having various functions and are therefore highly anticipated for application use in the fields of regenerative medicine, drug discovery, and the like. Cell culture apparatuses are used for culturing cells such as iPS cells and ES cells.

### BRIEF DESCRIPTION OF THE DRAWINGS

FIG. 1 is a schematic diagram illustrating an exemplary configuration of a cell culture apparatus according to a first embodiment;

FIG. 2 is a drawing for explaining an example of an operation performed by a specimen placement unit according to the first embodiment;

FIG. 3 is a flowchart illustrating an example of processes performed by a controlling apparatus of the cell culture apparatus according to the first embodiment;

FIG. 4 is a schematic diagram illustrating an exemplary configuration of a cell culture apparatus according to a second embodiment;

FIG. 5 is a drawing for explaining an example of an operation to export cell specimens according to the second embodiment;

FIG. 6 is a flowchart illustrating an example of processes in a producing step performed by a controlling apparatus of the cell culture apparatus according to the second embodiment; and

FIG. 7 is a flowchart illustrating an example of processes in an inspecting step performed by the controlling apparatus of the cell culture apparatus according to the second embodiment.

### DETAILED DESCRIPTION

A cell culture apparatus according to an embodiment includes a culture chamber, a holding unit, and processing circuitry. In the culture chamber, a biological specimen is cultured. The holding unit is provided in the culture chamber and configured to hold the biological specimen in such a manner that the position thereof in the culture chamber is movable. The processing circuitry is configured to control the holding unit so as to move the position of the biological specimen within the culture chamber, in a culture period for culturing the biological specimen.

Exemplary embodiments of a cell culture apparatus and a cell culture method will be explained in detail below, with reference to the accompanying drawings. Further, the cell culture apparatus and the cell culture method of the present application are not limited to the embodiments described below. Further, it is possible to combine any of the embodiments with other embodiments or conventional techniques, as long as no conflict occurs in the substance. In the description below, some of the constituent elements that are the same as or similar to each other will be referred to by using the same reference characters, and duplicate explanations thereof will be omitted.

### First Embodiment

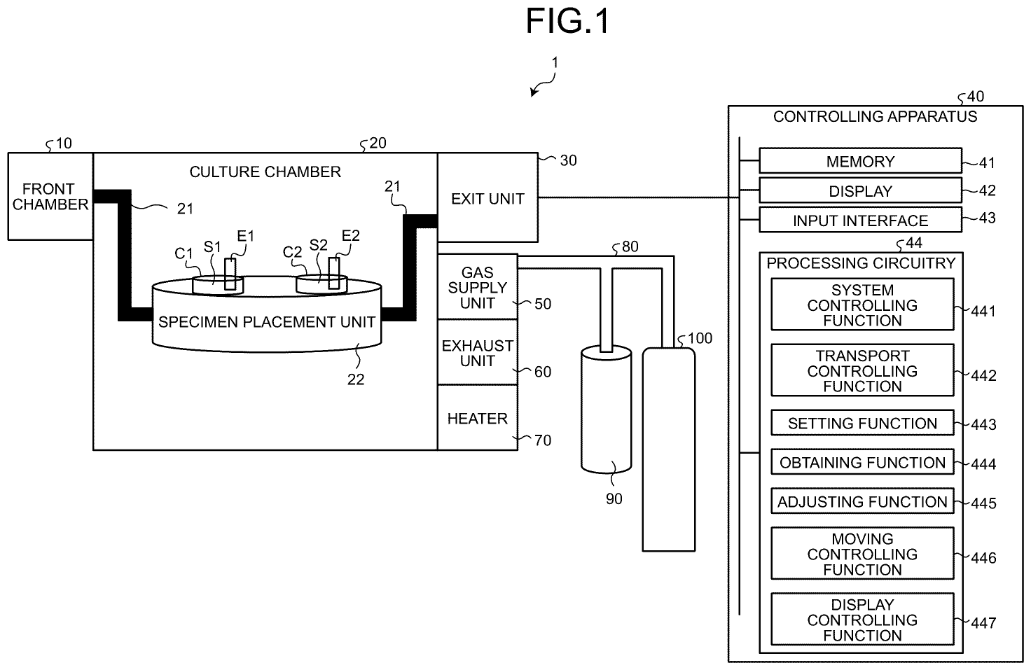

To begin with, a configuration of a cell culture apparatus 1 according to the present embodiment will be explained. The cell culture apparatus 1 is an apparatus configured to culture cells in a set culture environment (i.e., temperature, humidity, carbon dioxide concentration, and/or the like in the cell culture apparatus). FIG. 1 is a schematic diagram illustrating an exemplary configuration of the cell culture apparatus 1 according to the present embodiment.

The cell culture apparatus 1 includes a front chamber 10, a culture chamber 20, an exit unit 30, a controlling apparatus 40, a gas supply unit 50, an exhaust unit 60, a heater 70, a supply path 80, a water storage tank 90, and a CO2 tank 100.

FIG. 1 illustrates an example in which, a cell specimen S1 in a culture container C1 and a cell specimen S2 in a culture container C2 are cultured in the culture chamber 20. In the following description, when not being particularly differentiated from each other, the culture containers C1 and C2 may simply be referred to as culture containers C. Further, when not being particularly differentiated from each other, the cell specimens S1 and S2 may simply be referred to as cell specimens S. The cell specimens S are examples of the biological specimen.

The front chamber 10 is a space for placing the cell specimens S to be cultured. For example, in the front chamber 10, cell specimens held in the culture containers C are placed. To the culture containers C, Integrated Circuit (IC) chips such as Radio Frequency Identification (RFID) tags recording specimen IDs for identifying the cell specimens are attached. Alternatively, code symbols such as barcodes or two-dimensional codes may be attached, instead of the IC chips.

Further, provided in the front chamber 10 is a placement sensor (not illustrated) configured to detect the placement of any of the culture containers. As the placement sensor, it is possible to use a publicly-known sensor such as an infrared sensor, a weight sensor, or an electrostatic capacitive sensor. When a user places a culture container C in the placement space in the front chamber 10, the sensor is configured to detect placement of the cell specimen S.

Further, the front chamber 10 has a reading unit (not illustrated) configured to read information from the IC chip or the like. When the sensor detects the placement of the cell specimen S, the reading unit is configured to read information such as a specimen ID stored in the IC chip or the like attached to the culture container C holding the cell specimen S. After that, the culture container C is transported to the inside of the culture chamber 20 by a transport mechanism 21 (explained later).

The culture chamber 20 is a space in which the cell specimens S are cultured. For example, the culture chamber 20 is configured to maintain a culture environment having a temperature, a humidity level, and/or the like set by the user. The culture chamber 20 includes a transport mechanism 21 and a specimen placement unit 22. Although FIG. 1 depicts the culture container C1 and the culture container C2 in the specimen placement unit 22, the quantity of the culture containers C placed in the specimen placement unit 22 is not limited to two.

The transport mechanism 21 is a mechanism configured to transport the culture containers C. The transport mechanism 21 is a belt conveyor or the like, for example. Possible examples of the transport mechanism 21 are not limited to the belt conveyor. For example, under control of processing circuitry 44 (explained later), the transport mechanism 21 is configured to transport the culture containers C placed in the front chamber 10 to the inside of the culture chamber 20. Further, for example, the transport mechanism 21 is configured to place the culture containers C transported to the inside of the culture chamber 20 to the specimen placement unit 22. Further, for example, the transport mechanism 21 is configured to transport any of the culture containers C of which a culture time period (hereinafter, “culture period”) has finished, to the exit unit 30.

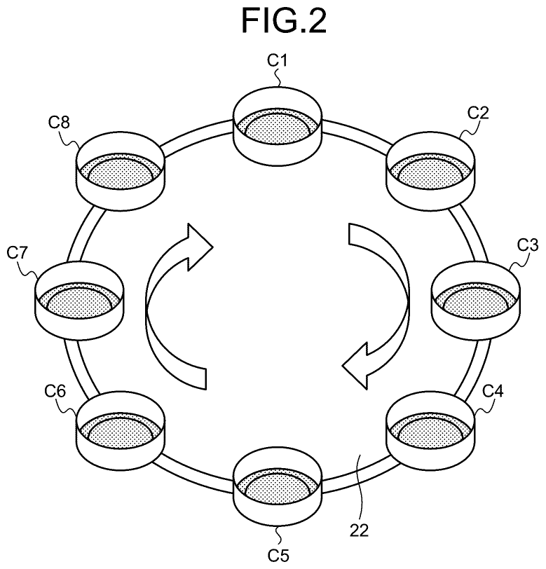

The specimen placement unit 22 is provided in the culture chamber 20 and is configured to movably hold the culture containers C (cell specimens). The specimen placement unit 22 is an example of the holding unit. The specimen placement unit 22 is provided with a plurality of culture container placement positions for placing the culture containers C. In the following sections, an operation of the specimen placement unit 22 will be explained, with reference to FIG. 2. FIG. 2 is a drawing for explaining an example of the operation performed by the specimen placement unit 22. FIG. 2 illustrates an example in which the culture containers C1 to C8 are placed in the culture container placement positions within the specimen placement unit 22.

As illustrated in FIG. 2, the specimen placement unit 22 is configured to rotate and move in the direction indicated by the arrows, under control of the processing circuitry 44. Accordingly, the culture containers C placed in the specimen placement unit 22 rotate and move in the culture chamber 20. As explained herein, the culture chamber 20 is configured to culture the cell specimens in the culture containers C, while the culture containers C are rotated and moved within the culture chamber 20.

Next, reasons why the cell specimens S are cultured while being rotated and moved will be explained. For example, in an example of a culture apparatus of a certain type in which the culture chamber is provided with a door so that the user puts in and takes out the culture containers C by opening and closing the door, the environment in the apparatus such as temperature, humidity, carbon dioxide concentration may vary among placement positions within the culture apparatus (e.g., depending on whether the placement position of each culture container is near the door or away from the door). In other words, there is a possibility that the environment in the culture chamber may be non-uniform.

Further, because an airflow occurs due to the opening and closing of the door, the environment in the culture chamber may become non-uniform depending on frequency of opening and closing the door. In addition, also depending on the quantity of the culture containers placed in the culture chamber and/or how many culture containers are piled up together, there is a possibility that the airflow in the apparatus may vary, and the environment in the culture chamber may become non-uniform. As a result, when the cell specimens are placed at the same locations in the culture apparatus for a long period of time, even when the cell specimens of the same type start being cultured at mutually the same time, there may be differences in properties such as colony formation or growing degrees of the cells.

To cope with the circumstances described above, the cell culture apparatus 1 according to the present embodiment is configured to be able to uniformize the culture environment, by performing culturing processes while moving the cell specimens S within the culture chamber 20.

For example, in the state illustrated in FIG. 2, let us discuss a situation where, in the culture chamber 20, the temperature is higher on the side where the culture container C7 is positioned and is lower on the side where the culture container C3 is positioned. In this situation, because the specimen placement unit 22 is configured to rotate and move, the culture containers C6 to C8 placed in the positions having higher temperatures will move, over the course of time, to the side having lower temperatures.

Similarly, the culture containers C2 to C4 placed in the positions having lower temperatures will move to the side having higher temperatures. Consequently, it is possible to lower the possibility of having the properties vary among the cell specimens S due to the non-uniformity of the culture environment.

In this situation, the rotating direction of the specimen placement unit 22 is not limited to the direction indicated in the example of FIG. 2. Further, the method for moving the specimen placement unit 22 is not limited to the rotation movement. For example, another arrangement is also acceptable in which a wheel is provided at each of the culture container placement positions in the specimen placement unit 22, so that the culture container C placed in each of the culture container placement positions is able to move, on the wheel, to an arbitrary position within the culture chamber 20.

Returning to the description of FIG. 1, sensors E1 and E2 are provided, respectively, in the placement positions of the culture containers C1 and C2 in the specimen placement unit 22. In the following sections, when not being particularly distinguished from each other, the sensors E1 and E2 may simply be referred to as sensors E. The sensors E are configured to move in the culture chamber 20 together with the culture containers C placed in the placement positions. The sensors E are configured to detect temperatures, humidity levels, and carbon dioxide concentration levels, for example. The processing circuitry 44 is configured to adjust the culture environment and to adjust moving speed of the specimen placement unit 22 in accordance with detection results of the temperatures, the humidity levels, and the carbon dioxide concentration levels. These processes will be explained later.

Further, the sensors E may each be structured with two or more sensors. Although the two sensors are provided in FIG. 1, the quantity of the sensors E is not limited to two. In another example, a single sensor E may be provided at the center of the specimen placement unit 22. In yet another example, as many sensors E as the quantity of the placement positions of the culture containers C may be provided in positions corresponding to the placement positions of the culture containers.

The exit unit 30 is a space into which any of the culture containers C holding a cell specimen S of which the culture period has finished is exported. For example, any of the culture containers C holding a cell specimen S of which the culture period set by the user has elapsed is exported by the transport mechanism 21 from the specimen placement unit 22 to the exit unit 30.

The controlling apparatus 40 is an information processing apparatus configured to integrally control the entirety of the cell culture apparatus 1. For example, it is possible to realize the controlling apparatus 40 by using a Personal Computer (PC), a workstation, or the like. A configuration of the controlling apparatus 40 will be explained later.

The gas supply unit 50 is a mechanism for supplying gas (or liquid) to the inside of the culture chamber 20. The gas supply unit 50 may include a rotating fan or the like. The gas supply unit 50 is used for adjusting the culture environment such as temperature, humidity, carbon dioxide concentration, and/or the like.

The exhaust unit 60 is a mechanism for exhausting gas (or liquid) from the inside of the culture chamber 20. The exhaust unit 60 may include a rotating fan or the like. The exhaust unit 60 is used for adjusting the culture environment such as temperature, humidity, carbon dioxide concentration, and/or the like.

The heater 70 is configured to adjust the temperature in the culture chamber 20. For example, under the control of the processing circuitry 44, the heater 70 is configured to adjust the temperature in the culture chamber 20, on the basis of a culture temperature set by the user and the temperatures detected by the sensors E.

The supply path 80 is a supply path for supplying the gas (or liquid) to the inside of the culture chamber 20. For example, the supply path 80 supplies humidifying water for adjusting the humidity in the culture chamber 20 or carbon dioxide for adjusting the carbon dioxide concentration in the culture chamber 20, to the inside of the culture chamber 20 via the gas supply unit 50.

The water storage tank 90 is a tank for storing the humidifying water for adjusting the humidity in the culture chamber 20. To the water storage tank 90, a pump or the like for pumping out the humidifying water in the water storage tank 90 is connected. For example, the processing circuitry 44 is configured to control operations of the pump or the like, so that the humidifying water is supplied from the water storage tank 90 to the inside of the culture chamber 20 via the gas supply unit 50.

The CO2 tank 100 is a tank filled with carbon dioxide for adjusting the carbon dioxide concentration in the culture chamber 20. The CO2 tank 100 has a valve or the like that can electronically be controlled. For example, the processing circuitry 44 is configured to control opening and closing operations of the valve or the like, so that the carbon dioxide is supplied from the CO2 tank 100 to the inside of the culture chamber 20 via the gas supply unit 50.

Next, a configuration of the controlling apparatus 40 will be explained. The controlling apparatus 40 includes a memory 41, a display 42, an input interface 43, and the processing circuitry 44. Data communication between the memory 41, the display 42, the input interface 43, and the processing circuitry 44 is performed via a bus.

The memory 41 is a storage apparatus such as a Hard Disk Drive (HDD), a Solid State Drive (SSD), or an integrated circuit storage apparatus configured to store therein various types of information. For example, the memory 41 is configured to store therein information about the cell specimens including the specimen IDs.

The memory 41 may be a drive apparatus configured to read and write various types of information from and to a portable storage medium such as a Compact Disc (CD), a Digital Versatile Disc (DVD), or a flash memory, or a semiconductor memory element such as a Random Access Memory (RAM). Further, a save area of the memory 41 may be inside the controlling apparatus 40 or may be inside an external storage apparatus connected to the controlling apparatus 40 via a network.

The memory 41 is configured to store therein various types of programs according to the present embodiment. For example, the memory 41 is configured to store therein a program related to execution of a system controlling function 441, a transport controlling function 442, a setting function 443, an obtaining function 444, an adjusting function 445, a moving controlling function 446, and a display controlling function 447 implemented by the processing circuitry 44.

The display 42 is configured to display various types of information. For example, the display 42 is configured to output a display screen indicating culture statuses of the cell specimens and being generated by the processing circuitry 44, as well as a Graphical User Interface (GUI) for receiving various types of operations from the user, and/or the like.

For example, as the display 42, it is possible to use, as appropriate, a Liquid Crystal Display (LCD), a Cathode Ray Tube (CRT) display, an Organic Electroluminescence Display (OELD), a plasma display, or other arbitrary displays.

Further, the display 42 may be provided on the outside of the culture chamber 20 or the like. Further, the display 42 may be of a desktop type or may be configured by using a tablet terminal or the like capable of wirelessly communicating with the main body of the controlling apparatus 40.

The input interface 43 is configured to receive various types of input operations from the user, to convert the received input operations into electrical signals, and to output the electrical signals to the processing circuitry 44. For example, the input interface 43 is configured to receive, from the user, culture conditions of the cell specimens and the like.

As the input interface 43, for example, it is possible to use, as appropriate, a mouse, a keyboard, a trackball, a switch, a button, a joystick, a touchpad, a touch panel display, or the like.

In the present embodiment, the input interface 43 does not necessarily need to include physical operation component parts such as a mouse, a keyboard, a trackball, a switch, a button, a joystick, a touchpad, a touch panel display, and/or the like.

For instance, possible examples of the input interface 43 include electrical signal processing circuitry configured to receive an electrical signal corresponding to an input operation from an external input mechanism provided separately from the apparatus and to output the electrical signal to the processing circuitry 44.

Further, the input interface 43 may be provided on the outside of the culture chamber 20 or the like. Further, the input interface 43 may be configured by using a tablet terminal or the like capable of wirelessly communicating with the main body of the controlling apparatus 40.

The processing circuitry 44 is configured to control operations of the entirety of the cell culture apparatus 1, in accordance with the electrical signals of the input operations output from the input interface 43.

For example, as hardware resources, the processing circuitry 44 includes a processor such as a Central Processing Unit (CPU), a Micro Processing Unit (MPU), or a Graphics Processing Unit (GPU) and memory elements such as a ROM, a RAM, and/or the like. The processing circuitry 44 is configured to implement the system controlling function 441, the transport controlling function 442, the setting function 443, the obtaining function 444, the adjusting function 445, the moving controlling function 446, and the display controlling function 447 by employing the processor that executes a program loaded into the memory.

The functions 441 to 446 do not necessarily need to be realized by the single piece of processing circuitry. It is also acceptable to structure processing circuitry by combining together a plurality of independent processors so that the functions 441 to 446 are realized as a result of the processors executing the program.

The system controlling function 441 is configured to control operations of the entirety of the cell culture apparatus 1. For example, the system controlling function 441 is configured to integrally control functional units of the processing circuitry 44, on the basis of setting input operations regarding the culture environment (temperature, humidity, carbon dioxide concentration, and/or the like) and the number of days of culturing (the culture period) received from the user via the input interface 43. The functional units of the processing circuitry 44 are configured to execute various types of processing, under the control of the system controlling function 441.

The transport controlling function 442 is configured to control the transport of the cell specimens S. For example, upon detection by the placement sensor that a culture container C holding a cell specimen S is placed in the front chamber 10, the transport controlling function 442 is configured to control the transport mechanism 21 so as to transport the culture container C to the inside of the culture chamber 20.

Further, for example, the transport controlling function 442 is configured to control the transport mechanism 21 so as to place the culture container C transported to the inside of the culture chamber 20, into a placement position for the culture container C provided in the specimen placement unit 22. Further, for example, the transport controlling function 442 is configured to control the transport mechanism 21 so as to transport the culture container C holding a cell specimen S of which the culture period set by the user has elapsed, from the placement position for the culture container C in the specimen placement unit 22 to the exit unit 30.

The setting function 443 is configured to set the culture environment of the culture chamber 20. For example, on the basis of the culture environment received by the system controlling function 441 from the user via the input interface, the setting function 443 is configured to set a temperature, a humidity level, and a carbon dioxide concentration level inside the culture chamber 20. Further, for example, on the basis of the number of days of culturing received by the system controlling function 441 from the user via the input interface, the setting function 443 is configured to set the culture period during which each cell specimen S is to be cultured.

The obtaining function 444 is configured to obtain sensing results from the sensors E. For example, the obtaining function 444 is configured to obtain the sensing results detected by the sensors E and indicating temperatures, humidity levels, and carbon dioxide concentration levels.

The adjusting function 445 is configured to adjust the environment in the culture chamber 20. For example, the adjusting function 445 is configured to adjust the temperature in the culture chamber 20 by controlling turning ON/OFF of the heater 70, on the basis of the temperature set by the setting function 443 and the temperatures detected by the sensors E and obtained by the obtaining function 444.

Further, for example, in place of the above or in addition to the above, the adjusting function 445 may be configured to adjust the temperature in the culture chamber 20, by adjusting at least one selected from between the amount of air supplied from the gas supply unit 50 to the inside of the culture chamber 20 and the amount of air exhausted from the exhaust unit 60.

Further, for example, the adjusting function 445 is configured to control the pump or the like connected to the water storage tank 90, on the basis of the humidity level set by the setting function 443 and the humidity levels detected by the sensors E and obtained by the obtaining function 444. Further, the adjusting function 445 is configured to adjust the humidity inside the culture chamber 20, by supplying the humidifying water in the water storage tank 90 to the inside of the culture chamber 20 via the gas supply unit 50.

Further, for example, in addition to the above, the adjusting function 445 may be configured to adjust the humidity in the culture chamber 20, by adjusting at least one selected from between the amount of humidifying water supplied from the gas supply unit 50 to the inside of the culture chamber 20 and the amount of air exhausted from the exhaust unit 60.

Further, for example, the adjusting function 445 is configured to control the opening and closing of a valve or the like of the CO2 tank 100, on the basis of a carbon dioxide concentration level set by the setting function 443 and the carbon dioxide concentration levels detected by the sensors E and obtained by the obtaining function 444. Further, the adjusting function 445 is configured to adjust the carbon dioxide concentration in the culture chamber 20 by supplying the carbon dioxide in the CO2 tank 100 to the inside of the culture chamber 20 via the gas supply unit 50.

Further, for example, in addition to the above, the adjusting function 445 may be configured to adjust the carbon dioxide concentration in the culture chamber 20, by adjusting at least one selected from between the amount of carbon dioxide supplied from the gas supply unit 50 to the inside of the culture chamber 20 and the amount of air exhausted from the exhaust unit 60. Further, although the adjusting function 445 in the present embodiment is configured to adjust the temperature, the humidity, and the carbon dioxide concentration in the culture chamber 20, the adjusting function 445 may be configured to adjust one or two of these factors.

The moving controlling function 446 is configured to control the operations of the specimen placement unit 22, so as to move the positions of the cell specimens S within the culture chamber 20, in the culture period for culturing the cell specimens S. For example, by controlling the specimen placement unit 22 on the basis of the temperatures, humidity levels, and the carbon dioxide concentration levels detected by the sensors E, the moving controlling function 446 is configured to move the culture containers C during the culture period, in particular, even outside the timing for taking out and putting in the culture containers C, so that the culture environment becomes uniform among the culture containers C holding the cell specimens S.

In an example, by referring to a history of sensing results of the sensors E respectively corresponding to the culture containers C (cell specimens S) placed in the culture container placement positions within the specimen placement unit 22 and being stored in the memory 41 or the like, the moving controlling function 446 is configured to control arrangement positions of the culture containers C in the culture chamber 20, so that the culture environment is uniform among the cell specimens S.

In that situation, the moving controlling function 446 is configured to detect culture environment differences among the cell specimens S, on the basis of the history of the sensing results of the sensors E. Further, the moving controlling function 446 is configured to control the rotation speed of the specimen placement unit 22 and to adjust the arrangement positions of the culture containers C in the culture chamber 20 so that the differences are eliminated.

In this situation, the moving controlling function 446 may rotate and move the culture containers C holding the cell specimen S in the set culture period, at a rotation speed set by the user. In that situation, the moving controlling function 446 is configured to rotate and move the specimen placement unit 22 continuously at the rotation speed set by the user throughout the culture period, regardless of the sensing results from the sensors E.

Further, the rotation of the culture containers C does not necessarily need to be continuous. As long as the rotation lasts throughout the culture period, the rotation may be intermittent. For example, in the culture period, the rotation may be carried out at a prescribed angle once every prescribed period of time. Further, the rotation of the culture containers C does not necessarily need to be continued throughout the entirety of the culture period.

The display controlling function 447 is configured to cause the display 42 to display various types of information. For example, the display controlling function 447 is configured to cause the display 42 to display a setting input screen for the culture environment. For example, the setting input screen for the culture environment may be a screen for receiving, from the user, inputs of a temperature, a humidity level, a carbon dioxide concentration level, a culture period, and/or the like.

Further, for example, the display controlling function 447 is configured to cause the display 42 to display a display screen indicating culture statuses of the cell specimens S. For example, the display screen indicating the culture statuses of the cell specimens S may be a screen for displaying: the specimen IDs of the cell specimens S; names of the cell specimens S; culture periods, elapsed periods since the start of the culturing process; temperatures, humidity levels, and carbon dioxide concentration levels that have been set; a temperature, a humidity level, and a carbon dioxide concentration level in the current position of each cell specimen S, and/or the like. In this situation, the names of the cell specimens S are, for example, stored in the memory 41 while being kept in correspondence with the specimen IDs.

Further, for example, the display controlling function 447 is configured to display a culture completion screen indicating that the culturing process of a cell specimen S has been completed when the culture period thereof has finished and the culture container C is exported to the exit unit 30. For example, the culture completion screen may be a screen for displaying the specimen IDs of the cell specimens S, the names of the cell specimens S, the dates/times of the end of the culturing processes, a message indicating that a culturing process has finished, and/or the like.

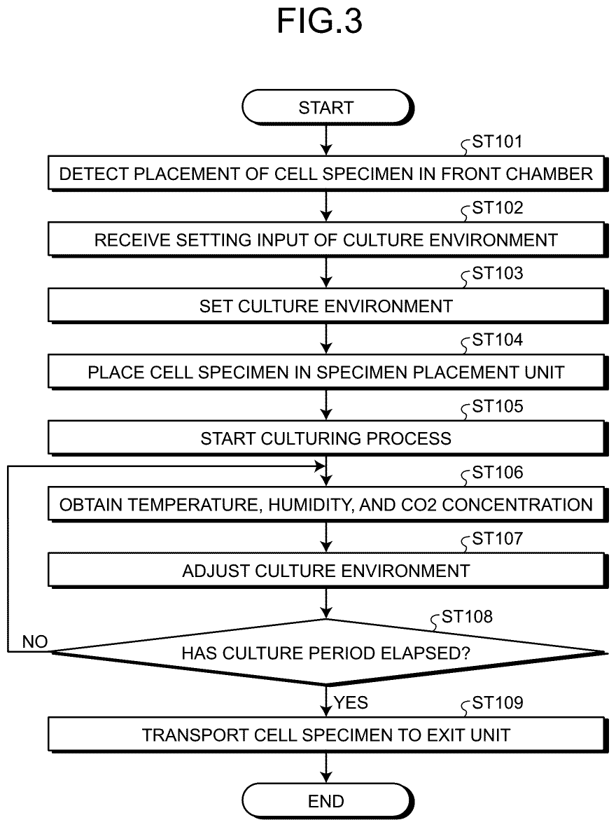

Next, processes performed by the controlling apparatus 40 of the cell culture apparatus 1 according to the present embodiment will be explained. FIG. 3 is a flowchart illustrating an example of the processes performed by the controlling apparatus 40 of the cell culture apparatus 1 according to the first embodiment.

To begin with, the transport controlling function 442 detects that a cell specimen S is placed in the front chamber 10 (step ST101). For example, when the placement sensor provided in the front chamber 10 detects an object, the transport controlling function 442 detects that the cell specimen S is placed in the front chamber 10.

When the placement of the cell specimen S in the front chamber 10 is detected, the reading unit provided in the front chamber 10 reads information about the specimen ID and the like stored in the IC chip or the like attached to the culture container C. The read information is output to the system controlling function 441.

By referring to cell specimen information stored in the memory 41 and keeping the specimen IDs in correspondence with the names of the cell specimens S, the system controlling function 441 is able to specify the cell specimen S placed in the front chamber 10. Further, the display controlling function 447 causes the display 42 to display the setting input screen for the culture environment.

Subsequently, the system controlling function 441 receives a setting input of the culture environment from the user (step ST102). For example, the system controlling function 441 receives, from the user, the setting input about a temperature, a humidity level, a carbon dioxide concentration level, a culture period, and/or the like. After that, on the basis of the specifics of the input received from the user at step ST102, the setting function 443 sets the culture environment of the cell specimen S (step ST103).

After that, the transport controlling function 442 places the cell specimen S in the specimen placement unit 22 (step ST104). For example, the transport controlling function 442 controls the transport mechanism 21 so as to transport the culture container C from the front chamber 10 to the inside of the culture chamber 20. After that, the transport controlling function 442 places the culture container C transported to the inside of the culture chamber 20, within the specimen placement unit 22.

Subsequently, the system controlling function 441 starts culturing the cell specimen S by integrally controlling the functional units of the processing circuitry 44 (step ST105). For example, in collaboration with the moving controlling function 446, the system controlling function 441 cultures the cell specimen S in the culture container C, while rotating and moving the specimen placement unit 22, in the culture period set at step ST103.

In this situation, the moving controlling function 446 may control the rotation moving speed of the specimen placement unit 22, in accordance with a rotation speed set by the user in advance or may control the rotation speed in accordance with the information indicating the temperature, the humidity, and the carbon dioxide concentration in the culture chamber 20 obtained at step ST106 (explained later).

Further, the obtaining function 444 obtains the information indicating the temperature, the humidity, and the carbon dioxide concentration in the culture chamber 20 (step ST106).

For example, the obtaining function 444 obtains the sensing results indicating the temperature, the humidity, and the carbon dioxide concentration, from the sensor E provided for each of the placement positions of the plurality of culture containers C included in the specimen placement unit 22. In this situation, although the present flowchart describes the process of obtaining the temperature, the humidity, and the carbon dioxide concentration in the culture chamber 20 as step ST106, the obtaining function 444 may continuously keep obtaining temperatures, humidity levels, and carbon dioxide concentration levels.

Subsequently, the adjusting function 445 makes adjustments so that the culture environment in the culture chamber 20 becomes the set culture environment (step ST107). For example, the adjusting function 445 adjusts the temperature by controlling the heater 70, on the basis of the temperature set at step ST103 and the temperature obtained at step ST106.

Further, for example, the adjusting function 445 adjusts the humidity by having humidifying water supplied from the water storage tank 90 on the basis of the humidity set at step ST103 and the humidity obtained at step ST106. Further, for example, the adjusting function 445 adjusts the carbon dioxide concentration by having carbon dioxide supplied from the CO2 tank 100, on the basis of the carbon dioxide concentration set at step ST103 and the carbon dioxide concentration obtained at step ST106.

In parallel with step ST107, the moving controlling function 446 may adjust the arrangement positions of the culture containers C in the culture chamber 20, by controlling the rotation speed of the specimen placement unit 22 so that the culture environment is uniform among the culture containers C holding the cell specimens S, on the basis of the sensing results that indicate the temperature, the humidity, and the carbon dioxide concentration and were obtained at step ST106.

Subsequently, the system controlling function 441 judges whether or not the culture period of the cell specimen S has finished (step ST108). For example, the transport controlling function 442 judges whether or not the culture period set at step ST103 has elapsed since the start of the culturing process. When the culture period has not elapsed (step ST108: No), the process proceeds to step ST106.

On the contrary, when the culture period has elapsed (step ST108: Yes), the transport controlling function 442 exports the cell specimen S to the exit unit 30 (step ST109), and the present processes are thus finished. For example, by controlling the transport mechanism 21, the transport controlling function 442 exports the culture container C holding the cell specimen S of which the culture period has finished, from the culture chamber 20 to the exit unit 30.

As explained above, the cell culture apparatus 1 according to the first embodiment is configured to control the specimen placement unit 22 that movably holds the cell specimens S, so as to move the positions of the cell specimens S within the culture chamber 20, in the culture period for culturing the cell specimens S.

As a result, in the culture period, the cell specimens S move in the culture chamber 20. Accordingly, the cell specimens S are prevented from being continuously placed in the same location for a long period of time. Consequently, even when the culture environment such as the temperature, the humidity, the carbon dioxide concentration, and/or the like in the culture chamber 20 is non-uniform, it is possible to uniformize the culture environment among the cell specimens S in the culture chamber 20. As a result of uniformizing the culture environment, it is possible, when a plurality of cell specimens S of mutually the same types are cultured for example, to avoid the situation where the properties of the cell specimens S vary depending on the differences in the culture environment. In other words, the cell culture apparatus 1 according to the first embodiment is able to level the impacts imposed on the cell specimens S by the culture environment.

Further, the cell culture apparatus 1 according to the first embodiment includes the sensors E that detect at least one selected from among the temperatures, the humidity levels, and the carbon dioxide concentration levels and is configured to adjust at least one selected from among the temperatures, the humidity levels, and the carbon dioxide concentration levels in the culture chamber 20, by adjusting at least one selected from among the amounts of air, humidifying water, carbon dioxide, and/or the like to be supplied to the inside of the culture chamber 20 and the amount of air to be exhausted from the inside of the culture chamber 20, on the basis of the sensing results from the sensors E.

Consequently, the cell culture apparatus 1 according to the first embodiment is able to uniformize the culture environment, after adjusting the culture environment inside the culture chamber 20 to be in a desirable state.

### Second Embodiment

In the first embodiment, the embodiment was explained in which the cell culture apparatus 1 includes the single culture chamber 20 for the cell producing step. As a second embodiment, another embodiment will be explained in which, in addition to the culture chamber for the producing step, a culture chamber for an inspecting step of the cells is provided.

In the following sections, differences from the above embodiment will primarily be explained. Detailed explanations of some of the features that are the same as those explained previously will be omitted. Furthermore, the embodiments explained below may be carried out individually or may be carried out in combination as appropriate.

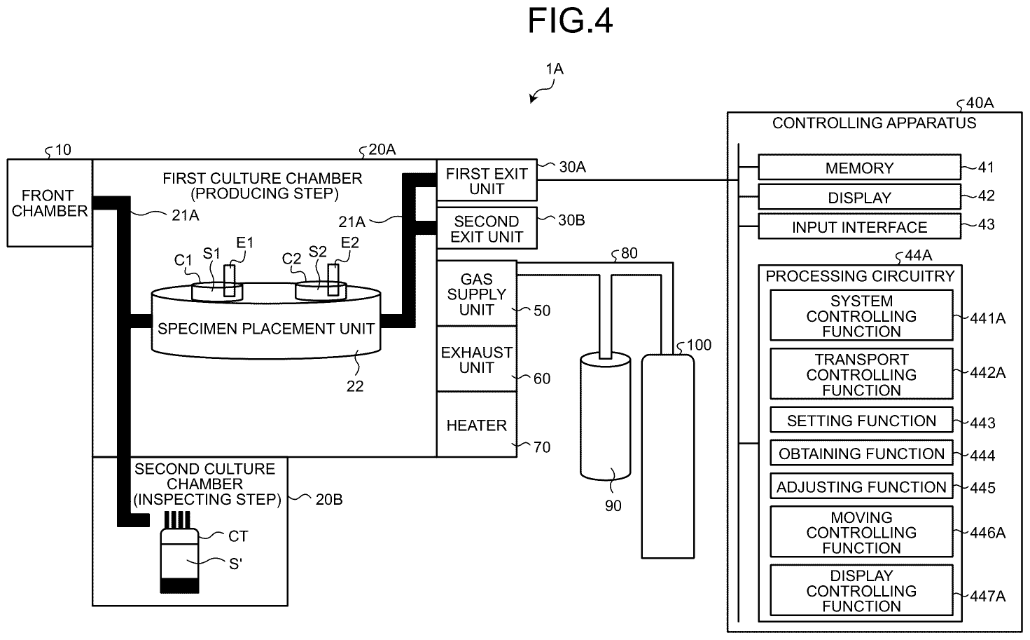

To begin with, a configuration of a cell culture apparatus 1A according to the second embodiment will be explained. FIG. 4 is a schematic diagram illustrating an exemplary configuration of the cell culture apparatus 1A according to the second embodiment.

The cell culture apparatus 1A according to the second embodiment, includes a front chamber 10A, a first culture chamber 20A, a second culture chamber 20B, a first exit unit 30A, a second exit unit 30B, a controlling apparatus 40A, the gas supply unit 50, the exhaust unit 60, the heater 70, the supply path 80, the water storage tank 90, and the CO2 tank 100. Because the gas supply unit 50, the exhaust unit 60, the heater 70, the supply path 80, the water storage tank 90, and the CO2 tank 100 have the same configurations as those in the first embodiment, explanations thereof will be omitted.

The front chamber 10A of the cell culture apparatus 1A according to the second embodiment includes a dispensing mechanism (not illustrated) configured to dispense cell specimens S. For example, the dispensing mechanism is configured to dispense the cell specimens S from a specimen storing container placed in the front chamber 10A, into a culture container C for the producing step and into an inspection container CT for the inspecting step. In the following sections, the cell specimen S dispensed in the inspection container CT shall be referred to as a cell specimen S′. The cell specimen S′ is an example of an inspection-purpose specimen.

Alternatively, another configuration is also acceptable in which the culture container C for the producing step is placed in the front chamber 10A, so that the cell specimen S′ is dispensed out of the cell specimen S in the culture container C into the inspection container CT.

Similarly to the first embodiment, IC chips or the like are attached to the culture container C and to the inspection container CT. In the second embodiment, the IC chips or the like have recorded therein information indicating specimen types (for the producing step or for the inspecting step), in addition to the specimen IDs. As a result, even when the appearance of the culture container C is similar to the appearance of the inspection container CT, it is possible to discriminate the two by reading the information from the IC chips or the like.

Under control of processing circuitry 44A, a transport mechanism 21A is configured to transport the culture container C in which the cell specimen S was dispensed, to the first culture chamber 20A. Similarly, the transport mechanism 21A is configured to transport the inspection container CT into which the cell specimen S′ was dispensed, to the second culture chamber 20B.

The first culture chamber 20A is a culture chamber for the culturing process in the producing step. Because the first culture chamber 20A has the same configuration as that of the culture chamber 20 in the first embodiment, explanations thereof will be omitted.

The second culture chamber 20B is a culture chamber for the culturing process in the inspecting step. Although not illustrated, it is assumed that the second culture chamber 20B also has the same configuration as that of the first culture chamber 20A. In this situation, the specimen placement unit 22 in the second culture chamber 20B does not necessarily need to include a rotating and moving mechanism.

For example, in the second culture chamber 20B, the cell specimen S′ dispensed in the inspection container CT to which a culture medium for a microorganism inspection was added is cultured for a prescribed period of time, so as to perform an inspection by checking to see whether or not bacteria grow on the culture medium. This inspection is carried out in parallel with the culturing process in the producing step in the first culture chamber 20A. With this configuration, it is possible to shorten the time to find out an inspection result, in comparison to the situation where the inspecting step is started after the producing step is finished.

The first exit unit 30A is an exit unit into which the culture container C is exported, when the inspecting step is determined to have no problems or when the culture period set before the completion of the inspecting step has finished.

The second exit unit 30B is an exit unit into which the culture container C is exported, when the inspecting step is determined to have a problem (e.g., when bacteria grew on the culture medium).

Next, a configuration of the controlling apparatus 40A will be explained. The controlling apparatus 40 includes the memory 41, the display 42, the input interface 43, and the processing circuitry 44A. Because the memory 41, the display 42, and the input interface 43 have the same configurations as those in the first embodiment, explanations thereof will be omitted.

The processing circuitry 44A is configured to implement a system controlling function 441A, a transport controlling function 442A, the setting function 443, the obtaining function 444, the adjusting function 445, a moving controlling function 446A, and a display controlling function 447A by employing a processor that executes a program loaded into a memory.

The system controlling function 441A is configured to receive an input of an inspection result from the user. The system controlling function 441A is configured to output the received inspection results to the transport controlling function 442A.

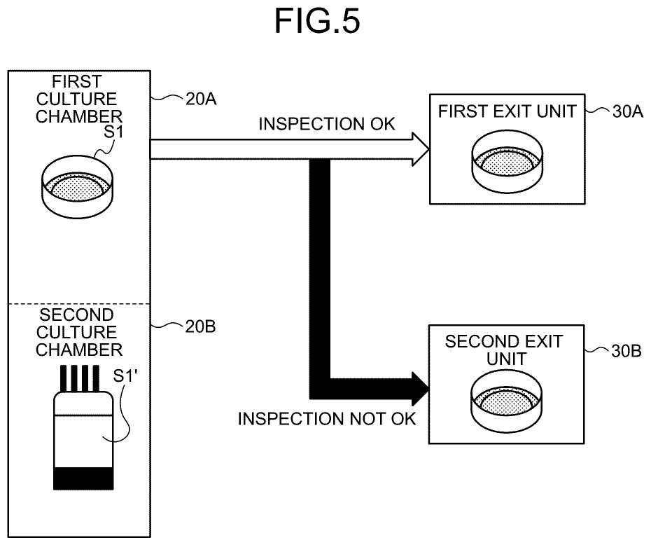

In accordance with the inspection result of the cell specimen S′, a transport controlling function 442A is configured to export the culture container C holding the cell specimen S to either the first exit unit 30A or the second exit unit 30B. In the following sections, an operation to export the cell specimen S will be explained, with reference to FIG. 5. FIG. 5 is a drawing for explaining an example of the operation to export the cell specimen S. FIG. 5 illustrates an example in which, in accordance with an inspection result of a cell specimen S1′ dispensed out of the cell specimen S1, a culture container C holding the cell specimen S1 is exported.

As illustrated in FIG. 5, for example, when an inspection result indicating that the inspecting step has no problem is output from the system controlling function 441A, the transport controlling function 442A is configured to control the transport mechanism 21A, so that the culture container C holding the cell specimen S is exported to the first exit unit 30A. In the present embodiment, it is assumed that, also when the culture period has finished before an inspection result becomes clear, the transport controlling function 442A is configured to export the culture container C holding the cell specimen S to the first exit unit 30A.

On the contrary, when an inspection result indicating that the inspecting step has a problem is output from the system controlling function 441A, the transport controlling function 442A is configured to control the transport mechanism 21A, so that the culture container C holding the cell specimen S is exported to the second exit unit 30B. The export of the culture container C to the second exit unit 30B may be performed after the producing step has finished or may be performed at the point in time when the inspecting step is found to have a problem. Further, an input may be received from the user indicating whether or not the culture container C is to be exported immediately.

As explained above, when the inspecting step is found to have a problem during the culturing process in the producing step, because the culture container C is exported from the second exit unit 30B different from normal situations, it is possible to avoid shipping cell specimens S not satisfying shipment quality as products. Further, because the cell specimen S′ used in the inspecting step is not exported from any of the exit units, it is possible to avoid mistaking the cell specimen S in the producing step for the cell specimen S′ in the inspecting step, and vice versa.

The moving controlling function 446A is configured to control the specimen placement unit in the second culture chamber 20B so that the position of the cell specimen S′ is moved within the second culture chamber 20B, in a specimen inspection culture period of the cell specimen S′. With this arrangement, it is possible to perform the inspection in an environment close to that of the first culture chamber 20A used for the producing step.

The display controlling function 447A is configured to cause the display 42 to display a result input screen for receiving an input of the inspection result. The system controlling function 441 is configured to receive the input of the inspection result from the user. The user determines an inspection result by checking the inspection container CT and inputs a determined result as the inspection result via the input interface 43. In that situation, the system controlling function 441 is configured to judge whether or not the inspecting step has a problem, on the basis of the input of the inspection result received from the user.

In the present embodiment, an example was explained in which the inspection result was determined as a result of the user visually checking the inspection container CT; however, possible methods for determining the inspection result are not limited to this example. For instance, the inspection result may be determined automatically, by providing an optical sensor and detecting turbidity of the cell specimen S′ in the inspection container CT. Alternatively, the inspection container CT may be imaged by using a camera, so as to automatically determine the inspection result on the basis of the captured image.

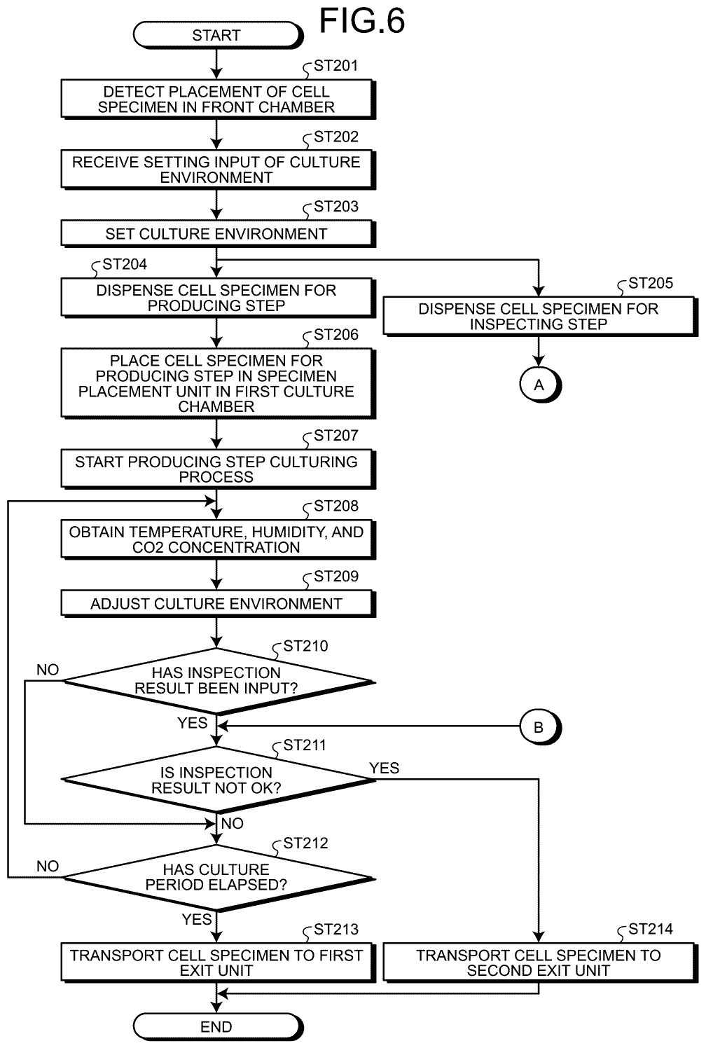

Next, processes performed by the controlling apparatus 40A of the cell culture apparatus 1A according to the present embodiment will be explained. At first, processes in the producing step will be explained. FIG. 6 is a flowchart illustrating an example of the processes in the producing step performed by the controlling apparatus 40A of the cell culture apparatus 1A according to the second embodiment. Because steps ST201 through ST203 are the same as steps ST101 through ST103 in FIG. 3, explanations thereof will be omitted.

When the culture environment is set at step ST203, the system controlling function 441A controls the dispensing mechanism so that a cell specimen S for the producing step is dispensed into the culture container C (step ST204). Further, the system controlling function 441A controls the dispensing mechanism so that a cell specimen S′ for the inspecting step is dispensed into the inspection container CT (step ST205). After the cell specimen S′ for the inspecting step is dispensed, the process proceeds to the processes in the inspecting step (A).

After the cell specimen S for the producing step is dispensed at step ST204, the transport controlling function 442A places the cell specimen S in the specimen placement unit 22 in the first culture chamber 20A, similarly to step ST104 in FIG. 3 (step ST206). Because steps ST207 through ST209 are the same as steps ST105 through ST107 in FIG. 3, explanations thereof will be omitted.

After the culture environment is adjusted at step ST209, the transport controlling function 442A judges whether or not the system controlling function 441A has received an input of an inspection result from the user (step ST210). For example, when an inspection result has been output from the system controlling function 441A, the transport controlling function 442A determines that an input of the inspection result is received from the user. On the contrary, for example, when no inspection result has been output from the system controlling function 441A, the transport controlling function 442A determines that no input of the inspection result has been received from the user.

When no input of the inspection result has been received from the user (step ST210: No), the process proceeds to step ST212. On the contrary, when an input of the inspection result has been received from the user (step ST210: Yes), the system controlling function 441A judges whether or not the inspecting step has a problem on the basis of the inspection result (step ST211).

When the inspecting step has a problem (when the inspection result indicates “NOT OK”) (step ST211: Yes), the transport controlling function 442A exports the cell specimen S in the first culture chamber 20A into the second exit unit 30B (step ST214), and the present process thus ends. For example, the transport controlling function 442 controls the transport mechanism 21, so that the culture container C holding the cell specimen S of which the inspection result was not OK is exported from the first culture chamber 20A into the second exit unit 30B.

On the contrary, when the inspecting step has no problem (when the inspection result indicates “OK”) (step ST211: No), the system controlling function 441A judges whether or not the culture period of the cell specimen S has finished, similarly to step ST108 in FIG. 3 (step ST212). When the culture period has not elapsed (step ST212: No), the process proceeds to step ST208.

On the contrary, when the culture period has elapsed (step ST212: Yes), the transport controlling function 442 exports the cell specimen S to the first exit unit 30A (step ST213), and the present process thus ends. For example, the transport controlling function 442 controls the transport mechanism 21, so as to export the culture container C holding the cell specimen S of which the culture period has finished, from the first culture chamber 20A into the first exit unit 30A.

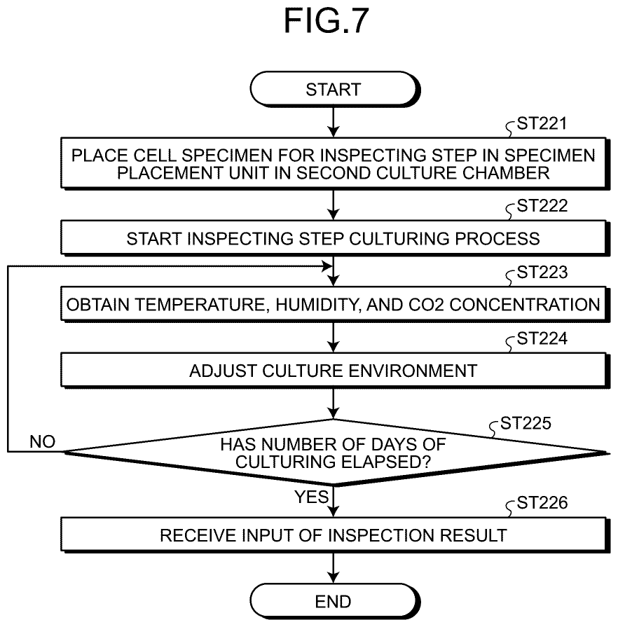

Next, processes in the inspecting step will be explained. FIG. 7 is a flowchart illustrating an example of the processes in the inspecting step performed by the controlling apparatus 40A of the cell culture apparatus 1A according to the second embodiment.

After step ST205 in FIG. 6, the transport controlling function 442A places the cell specimen S′ in the specimen placement unit in the second culture chamber 20B (step ST221). For example, the transport controlling function 442A controls the transport mechanism 21 so as to transport the inspection container CT from the front chamber 10 to the inside of the second culture chamber 20B. After that, the transport controlling function 442A places the inspection container CT transported to the inside of the second culture chamber 20B, into the specimen placement unit.

Subsequently, the moving controlling function 446A starts an inspection culturing process on the cell specimen S′ (step ST222). For example, the moving controlling function 446 cultures the cell specimen S′ in the inspection container CT while rotating and moving the specimen placement unit, in the culture period set at step ST203 in FIG. 6. Because steps ST223 through ST225 are substantially the same as steps ST106 through ST108 in FIG. 3, explanations thereof will be omitted.

When the culture period has elapsed at step ST225 (step ST225: Yes), the display controlling function 447A causes the display 42 to display an input screen for an inspection result. Subsequently, the system controlling function 441A receives, from the user, an input of the inspection result, via the displayed input screen for the inspection result and the input interface 43 (step ST226). After step ST226, the process proceeds to step ST211 in FIG. 6 (B).

As explained above, the cell culture apparatus 1A according to the second embodiment includes the second culture chamber 20B for performing the culturing process related to the inspecting step on the cell specimen S′ dispensed out of the cell specimen S. Accordingly, it is possible to perform the culturing process on the cell specimen S in the producing step, in parallel with the culturing process on the cell specimen S′ related to the inspecting step. Consequently, the cell culture apparatus 1A according to the second embodiment makes it possible to start the producing step and the inspecting step at the same time. It is therefore possible to make the finish time of the inspecting step earlier. Further, making the finish time of the inspecting step earlier lowers the possibility of having situations where, for example, it is learned that a product did not pass a bacteria-free test after the product is shipped. In addition, because the first culture chamber 20A is clearly separated from the second culture chamber 20B, it is possible to lower the risk of the user mistaking the cell specimen S in the producing step for the cell specimen S′ in the inspecting step, and vice versa.

It is possible to carry out any of the embodiments described above with an appropriate modification, by changing a part of the configurations or the functions of the apparatuses. Thus, modification examples of the above embodiments will be explained below, as other embodiments.

In the following sections, differences from the above embodiments will primarily be explained, and detailed explanations of some of the features that are the same as those explained previously will be omitted. Furthermore, the modification examples explained below may be carried out individually or may be carried out in combination as appropriate.

### First Modification Example

In the first and the second embodiments described above, an example was explained in which the moving controlling function 446 (446A) is configured to control the rotation speed of the specimen placement unit 22 so that the culture environment is uniform among the culture containers C holding the cell specimens S. Alternatively, the moving controlling function 446 (446A) may be configured to control the moving of the specimen placement unit 22 while making it possible to get an approximate understanding of how much time has elapsed since the start of the culturing process with respect to each of the cell specimens S placed in the specimen placement unit 22.

In the present modification example, for instance, the transport controlling function 442 (442A) is configured to place a cell specimen S in a position C1 in FIG. 2 at the start of a culturing process. Also, the moving controlling function 446 (446A) is configured, for example, to control the moving speed of the specimen placement unit 22, so that the cell specimen S that was placed in the position C1 in FIG. 2 at the start of the culturing process is present in a position C8 in FIG. 2 at the end of the culturing process. In an example, when the culture period is eight days, the moving controlling function 446 (446A) is configured to control the moving speed of the specimen placement unit 22 so that the cell specimen S moves from position C1 to position C8 over the eight days.

With this configuration, for example, the cell specimen S on the first day since the culturing process started is present between the position C1 and a position C2. The cell specimen S on the second day since the culturing process started is present between the position C2 and a position C3. Consequently, in the present modification example, the user is able to roughly understand the elapsed period of the cell specimen S since the start of the culturing process, by simply viewing the position of the cell specimen S.

### Second Modification Example

In the first and the second embodiments described above, an example was explained in which the plurality of culture containers C are placed in the specimen placement unit 22 without any other elements; however, it is also acceptable to place the plurality of culture containers C in the specimen placement unit 22 while being housed in capsules.

In the present modification example, the sensors E are provided so as to be able to detect the culture environment inside each of the capsules housing the respective culture containers C therein. With this arrangement, the sensing results from the sensors E reflect the culture environment of each of the culture containers C more accurately. Consequently, according to the present modification example, it is possible to manage the culture environment inside the culture chamber 20 (the first culture chamber 20A) in units of the culture containers C.

### Third Modification Example

In the first and the second embodiments described above, an example was explained in which the transport mechanism 21 (21A) is the belt conveyor. However, the transport mechanism 21 (21A) may be an electromagnetic induction coil or the like. When the culture containers C are transported by using an electromagnetic induction coil, because the component parts structuring the transport mechanism 21 (21A) do not get worn out, it is possible to lower the possibility of having dust, component part chippings, or the like, occurring inside the culture chamber 20 (the first culture chamber 20A).

According to at least one aspect of the embodiments described above, it is possible to level the impacts imposed on the cell specimens by the culture environment.

The term “processor” used in the above explanation denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)).

In this situation, instead of having the programs saved in a memory, it is also acceptable to directly incorporate the programs in the circuitry of one or more processors. In that situation, the one or more processors realize the functions by reading and executing the programs incorporated in the circuitry thereof. Further, the processors in the present embodiments do not each necessarily have to be structured as a single piece of circuitry. It is also acceptable to structure one processor by combining together a plurality of pieces of independent circuitry so as to realize the functions thereof.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell culture apparatus comprising:
a culture chamber in which a biological specimen is cultured;
a holding unit provided in the culture chamber and configured to hold the biological specimen in such a manner that a position thereof in the culture chamber is movable; and
processing circuitry configured to control the holding unit so as to move the position of the biological specimen within the culture chamber, in a culture period for culturing the biological specimen.

2. The cell culture apparatus according to claim 1, wherein the processing circuitry is configured to adjust an environment in the culture chamber.

3. The cell culture apparatus according to claim 2, wherein, as the environment, the processing circuitry is configured to adjust at least one selected from among temperature, humidity, and carbon dioxide concentration in the culture chamber.

4. The cell culture apparatus according to claim 2, further comprising:
a gas supply unit configured to supply gas from an outside to an inside of the culture chamber; and
an exhaust unit configured to exhaust gas from the inside to the outside of the culture chamber, wherein
the processing circuitry is configured to adjust the environment by adjusting at least one selected from between a supply amount of the gas from the gas supply unit and an exhaust amount of the gas from the exhaust unit.

5. The cell culture apparatus according to claim 2, further comprising:
a sensor configured to detect an index related to the environment in the culture chamber; and
an obtaining unit configured to obtain a sensing result from the sensor, wherein
the processing circuitry is configured to adjust the environment on a basis of the obtained sensing result.

6. The cell culture apparatus according to claim 5, wherein the processing circuitry is configured to adjust a moving speed of the holding unit on the basis of the obtained sensing result.

7. The cell culture apparatus according to claim 5, wherein the sensor is provided for the holding unit, and the processing circuitry is configured to move a position of the sensor within the culture chamber, together with the biological specimen.

8. The cell culture apparatus according to claim 1, wherein
a test-purpose specimen is placed in the culture chamber, and
the cell culture apparatus further comprises an inspecting unit configured to inspect the test-purpose specimen.

9. A cell culture method using a cell culture apparatus, wherein
the cell culture apparatus includes a culture chamber for culturing a biological specimen and a holding unit provided in the culture chamber and configured to movably hold the biological specimen, and
the cell culture method comprises controlling the holding unit so as to move a position of the biological specimen within the culture chamber, in a culture period for culturing the biological specimen.
